(19) United States
(12) Patent Application Publication
(10) Pub. No.: US 2021/0373655 A1
(43) Pub. Date: Dec. 2, 2021

(54) AUGMENTED REALITY-BASED SYSTEM FOR MEASURING AND TREATING VERGENCE DISORDERS AND METHOD OF USING THE SAME

(71) Applicant: HTS, Inc., Gold Canyon, AZ (US)

(72) Inventors: Rodney K. Bortel, Gold Canyon, AZ (US); Jeffrey Cooper, New York, NY (US)

(21) Appl. No.: 17/243,857

(22) Filed: Apr. 29, 2021

Related U.S. Application Data
(60) Provisional application No. 63/032,397, filed on May 29, 2020.

(51) Int. Cl. G06F 3/01; A61B 3/113; G02B 27/01

(52) U.S. Cl. CPC G06F 3/013; G02B 27/017; A61B 3/113

(57) ABSTRACT

A system and method for measuring and treating vergence disorders may include an augmented reality headset connected to a computing device for simultaneously presenting target images independently to a patient's eyes and independently tracking the position of each eye. The headset includes eye tracking technology for monitoring the orientation of the eyes. If the eyes are both on target, as determined by the eye tracking technology, then binocular single vision will occur, and if not, then diplopia will occur. A vergence demand may be created by incrementally moving the target images apart or together, until the vergence demand is too great for the patient to maintain SBV. Amplitudes of fusional vergence may be measured and stored by the computing device. The system may be utilized to perform therapeutic vergence treatment exercises to increase amplitudes of fusional vergence and measure and provide treatment for phoria and strabismus conditions.

### CROSS REFERENCE TO RELATED APPLICATION[S]

This application claims priority to U.S. Provisional Patent Application entitled “AUGMENTED REALITY-BASED SYSTEM FOR MEASURING AND TREATING VERGENCE DISORDERS AND METHOD OF USING THE SAME,” Ser. No. 63/032,397, filed May 29, 2020, the disclosure of which is hereby incorporated entirely herein by reference.

### BACKGROUND OF THE INVENTION

### Technical Field

This invention relates generally to vision therapy systems and particularly to an augmented reality-based system for measuring and treating vergence disorders. A method of using the system for measuring and treating vergence disorders is disclosed.

### State of the Art

Dysfunctions in binocular vision affect quality of vision and quality of life as it is an important socioeconomic issue in modern society. Ocular disorders are affecting people at increasing rates in the smartphone era, as more people are using electronic devices at close ranges for prolonged periods. Among disorders of binocular function, vergence insufficiency is the inability to converge or diverge the eyes smoothly and effectively to the object of interest and/or inability to maintain the vergence angle. Vergence orthoptic training (also called vision therapy) helps to alleviate symptoms and usually corrects the condition.

Embodiments of a system for measuring and treating vergence disorders may comprise a computing device coupled to a monitor screen and a user input device such as a joystick or gamepad. The computing device may be programmed to perform certain functions in response to user input from the user input device to the computing device.

The computing device may be programmed to present images or targets to the eyes, by means of a computer monitor, which contain first (simultaneous perception), second (flat fusion) or third (stereoscopic) degree targets. Each eye is presented its own target. This is accomplished by the use of special glasses that allow each eye to see a separate image. When second and third degree targets are presented to the patient's eyes, so that both eyes are pointed directly at the target, single binocular vision (SBV) will occur, and the patient will see the target images as a single fused image. If one of the patient's eyes is on target while the other is not, then either diplopia (double vision), suppression and/or anomalous retinal correspondence will occur.

With SBV occurring (two images overlapping so that they appear to be one image), various vergence demands may be created by the system incrementally moving the target images apart, inducing a divergence or convergence vergence demand. As the images are moved farther apart, the patient's eyes will maintain alignment (making the image appear to the viewer as one image) until the vergence demand becomes too great for the patient to maintain SBV. At this point, one of the patient's eyes will deviate from its target, resulting in diplopia (the appearance of two images rather than one), and the patient indicates to the computing device, by use of the user-input device, that separation has occurred (in other words, the patient is seeing two images rather than one). The amplitudes of fusional vergence (how large the deviation is when the eyes begin seeing two images) in various directions (i.e. separating the objects in various directions, such as horizontally, vertically, etc.), may thus be measured and stored by the computing device. In addition, embodiments of the system may be utilized to perform therapeutic vergence treatment exercises to increase amplitudes of fusional vergence.

It is also possible to use targets and subjective user feedback to determine other parameters associated with eye various eye functions. For example, first degree or “simultaneous perception” targets (i.e., targets with non-fusible features (meaning targets that are not identical such that if overlapped, they appear to be one single target), such as, for example, a bird presented to one eye and a birdcage to the other eye) may be presented to each eye and used to subjectively measure “phoria position” of the eyes (position of rest for each eye). This may be done by having the user move the bird until, in the user's perception, the bird is located in the cage (at which point the system operator may measure the phoria position). Similar subjective approaches may be employed (using subjective user input) to determine the “subjective angle” (the perceived alignment when each eye has the same visual direction of the target).

It should be appreciated that in all of the above, determination of when fusion (the two images appearing to be one image) is lost or gained, or the phoria position, or strabismus, or the subjective angle is completely based on the user feedback that is manually provided to the computing device responsive to the patient manipulating controls on the user-input device when the patient perceives that vergence has been lost. It is also dependent on the attention span of the patient (the patient observing precisely the point when fusion is lost or gained), and how quickly or slowly the patient provides the feedback to the user-input device. Consequently, both the accuracy and utility of the system are impacted by patient characteristics with respect to providing feedback to the system. More specifically, in many cases, patient feedback may be unreliable, particularly with young patients. For example, patients may provide incorrect feedback in an attempt to provide favorable responses to simply please the examiner or to end the procedure prematurely, or with infants who may be unable to provide feedback.

When patient feedback is employed to capture or evaluate these conditions, these visual responses to lack of fusion can only be determined by sophisticated patients who are able to adequately describe these phenomena, which in any case, as noted above, are subjective responses rather than objective responses.

Accordingly, what is needed is a reliable, objective means and system for determining when fusion has occurred, or has been lost, and for measuring phoria, strabismus, subjective angle, and other eye characteristics, and means and system for effective treatment of vision problems related to the occurrence of loss of fusion and other eye characteristics.

### SUMMARY OF THE INVENTION

The present invention relates to vision therapy systems and particularly to a system for measuring and treating vergence disorders and a method of using the system.

Embodiments of a system and method for measuring and treating vergence disorders may comprise a computing device coupled to an augmented reality headset. The headset includes equipment for determining and tracking eye position and movement (eye tracking technology). Specifically, the system is configured to be able to independently track the position and movement of each eye of a person wearing the headset. The computing device may be programmed to perform certain functions in response to user input to the computing device and/or receipt of communication signals from the headset, including headset data regarding the position of each of the user's eyes at any given point in time.

The computing device may be programmed to present first (simultaneous perception), second (flat fusion) or third (stereoscopic) degree images to the headset. Each eye is presented a separate target, although the targets may be positioned by the system such that the targets appear to the user to be only one target rather than two. The eye tracking function of the headset is used to determine and monitor the orientation and/or position of each of the patient's eyes independently. The system, utilizing this data in conjunction with the known positions of the images which are projected by it to each of the patient's eyes, is configured to determine whether the patient's eyes are directed to their corresponding target images. For second and third degree targets, the patient's eyes are both initially pointed directly at the target projected to each eye, and single binocular vision (SBV) occurs (the patient sees the target images as a single fused image rather than two separate images). In the event that one of the patient's eyes is on target while the other is not, then either diplopia (double vision), suppression and/or anomalous retinal correspondence will occur.

The system first determines that SBV is occurring using each eye's position data as communicated to the system from the headset, and the known position of the target for each eye that is being projected by the system (the system knows where the image is being projected for each eye). In other words, the system first determines that each eye is directed at its target, and captures the data with respect to both the position of each target, and of each eye with respect to its own target. Once SBV is confirmed (the system determines that each eye is directed at its own target), various vergence demands may be created by the system by incrementally moving the target images apart (either inducing a divergence or convergence vergence demand). As this occurs, the system continues to monitor the position of each target as presented to each eye independently, and each eye's position relative to its respective target, and captures and analyzes that data as it is gathered. The patient's eyes will maintain alignment (each eye being directed at its own target) until the vergence demand becomes too great for the patient to maintain SBV. At this point, one of the patient's eyes will deviate from its corresponding target, resulting in diplopia (the user seeing two images rather than one). The computing device recognizes when this occurs (as soon as one of the eyes deviates from its target). It determines and logs the exact point at which this occurs (loss of fusion) by analyzing the eye position and target position data to determine when at least one of the eyes is no longer aligned on the target. The amplitudes of fusional vergence (the amplitude at which fusion is lost) may thus be stored in the computing device for later use by doctors or the system itself. To be clear, the term “amplitude of fusional vergence” means the maximum amount of vergence measured while still maintaining exact alignment of the eyes. This term is well known by practitioners in the field of vision care, and may be quantified using a variety of terms, the most common of which is “prism diopters.” Those in the field will appreciate that because the system knows the position of the targets being projected to each eye, and the direction of gave of each eye (eye position), the amplitude of fusional vergence may be determined. FIG. 7 generally illustrates an example of how the amplitude of fusional vergence may be calculated when targets are moved from a point of fusion (sometimes referred to as the “Donder's Point” or “Donder's Line”) to a position where the targets are separated. For example, referring to FIG. 7, two boxes 72 (labeled A) are positioned such that they completely overlap each other, such that the two boxes appear to be one box A (72). A left eye 70 and a right eye 71 of a person look at the box A, with the line-of-sight 75 and line-of-sight 77 from the right eye and left eye, respectively, intersecting at box A, and forming the angle 79. Next, one box 72 is moved to the right, and one box is moved to the left, such that the boxes (now referred to as boxes 73 (labeled B) appear as shown in FIG. 7. At this point, the right eye 71 is looking in the direction of the left box 73, along line-of-sight 76, and the left eye 70 is looking in the direction of the right box 73 along line-of-sight 78. As can be seen in FIG. 7, the two lines-of-sight 76 and 78 intersect at 74 (labeled box C), forming angle 80, and the person appears to see the image 74 (box C) at the position shown in FIG. 7. By measuring the difference in the magnitudes of the angles 79 and 80, eye care providers can track how a person's eyes respond to movement in the boxes A (and B), and can also determine at which angle or point vergence is lost (the point at which the eyes are no longer viewing a single image, but are viewing two separate images). It should be appreciated that the noted vergence demand that is introduced in order to determine the amplitude of fusional vergence (gradually moving the targets apart from each other) may be created horizontally (e.g. creating left-to-right separation between the targets), vertically (e.g. creating top-to-bottom separation of the targets), or in any other direction, and that amplitudes of fusional vergence may be stored for each case. Importantly, the measurement of fusional vergence determined by this system and method is precise and objective, and completely independent of any manual user input from the patient. This is because the relevant metrics (position of each eye, position of each target, position of each eye relative to a reference point) are directly measured by the system itself without intermediate, subjective user input. In addition, embodiments of the system may be utilized to perform therapeutic vergence treatment exercises to increase amplitudes of fusional vergence (increase the amount by which the targets may be separated before fusion is lost).

In sum, the occurrence of SBV or loss of fusion, may be determined accurately and automatically by the system using the eye tracking technology of the headset, rather than depending on feedback from the patient. Furthermore, the system may provide treatment to patients using similar processes as those used to determine SBV or diplopia.

It should be appreciated that the system's ability to independently track and measure the eye movement and the position of each eye also allows for objective measurement of either a phoria (in layman's terms, where one's eye is directed when it is covered and not seeing anything) or strabismus (in layman's terms, when both eyes are fixating on a single target, but one of the eyes is actually not aligned with or looking at the target). Practitioners in the field recognize that in a strabismus situation, if the brain were not “blanking”, ignoring or suppressing the signal from the non-aligned eye, the patient would see double vision in a strabismus situation. It should be appreciated that in some cases, prior to using the system to measure or treat vergence disorders, it might be helpful to first measure any phoria or strabismus condition such that position of the targets projected to each eye can be adjusted for these conditions so that the patient initially attains fusion at the beginning of the vergence measurement/treatment session.

The system is also configured, as described in greater detail below, to objectively measure phoria and strabismus conditions, and provide for treatment of the same. More specifically, to measure phoria and strabismus conditions, images are projected to each eye. The system tracks and stores the position of each eye at this point. Next, the image is blanked (removed from display) from one of the eyes for a short period of time, and any movement of each of the eyes (including the eye's final resting position) is tracked and logged by the system. Using this independently-tracked final eye position data, and comparing it to the initial eye position data, the system can determine the existence (and magnitude) of a phoria or strabismus condition. Next, the same is done with the opposite eye blanked, the data is captured by the system, and the system determines the existence (and magnitude) of a phoria or strabismus condition for the opposite eyes. The eye care provider can then use this information to determine a treatment plan.

Finally, the system and methods above can be utilized in a “field of gaze” evaluation to determine vergence, phoria, and strabismus conditions for multiple gaze positions. In this embodiment, the system performs the same steps as above, but in various unique gaze positions known as motor fields, allowing the eye care provider to specifically identify issues with particular fields of gave and provide treatment accordingly.

The foregoing and other features and advantages of the present invention will be apparent from the following more detailed description of the particular embodiments of the invention, as illustrated in the accompanying drawings.

### BRIEF DESCRIPTION OF THE DRAWINGS

A more complete understanding of the present invention may be derived by referring to the detailed description and claims when considered in conjunction with the Figures, wherein like reference numbers refer to similar items throughout the Figures, and:

FIG. 1 is a block diagram of an augmented reality-based system for measuring and treating vergence disorders, according to an embodiment;

FIG. 2 is a diagram representing images displayed and presented independently to the left and right eyes of a patient on an augmented reality headset of an augmented reality-based system for measuring and treating vergence disorders, according to an embodiment;

FIG. 3 is a diagram representing images displayed and presented independently to the left and right eyes of a patient on an augmented reality headset of an augmented reality-based system for measuring and treating vergence disorders, according to an embodiment;

FIG. 4 is a diagram representing images displayed and presented independently to the left and right eyes of a patient on an augmented reality headset of an augmented reality-based system for measuring and treating vergence disorders, according to an embodiment;

FIG. 5 is a block diagram of steps of a method of using an augmented reality-based system for measuring and treating vergence disorders, according to an embodiment;

FIG. 6 is a block diagram of additional steps of a method of using an augmented reality-based system for measuring and treating vergence disorders, according to an embodiment;

FIG. 7 is a figure generally illustrating the measurement of an amplitude of fusional vergence;

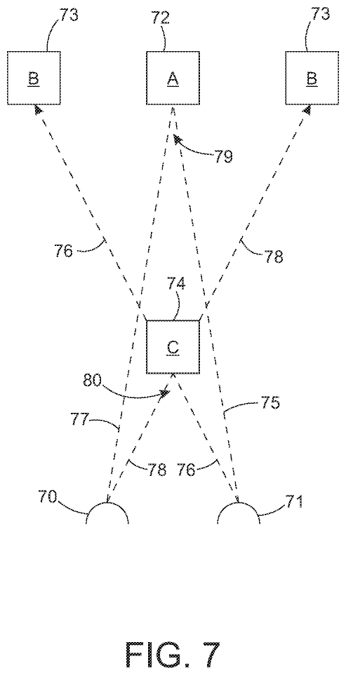

FIGS. 9a and 9b generally illustrate the determination, by the system, of a strabismus or phoria condition;

FIG. 10 generally illustrates an aspect of a “motor fields” determination by the system;

FIG. 11 is a block diagram of steps of a method of using an augmented reality-based system for determining and treating a strabismus or phoria condition of a patient; and

FIG. 12 is a block diagram of steps of a method of using an augmented reality-based system for completing a motor fields vision evaluation and treatment.

### DETAILED DESCRIPTION OF EMBODIMENTS OF THE INVENTION

As discussed above, embodiments of the present invention relate to vision measurement and therapy systems and particularly to an augmented reality-based system for measuring and treating vergence disorders, and methods of using the system.

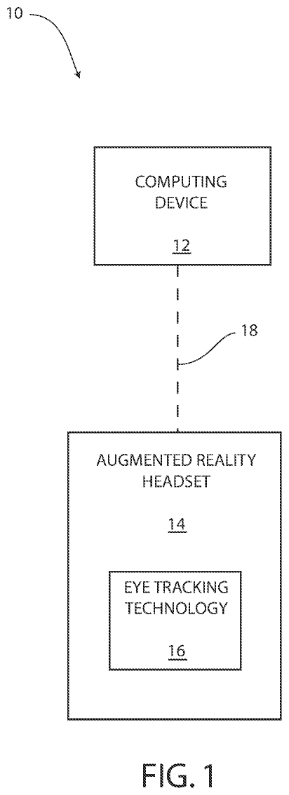

Referring to the Drawings, as shown in FIG. 1, embodiments of a system 10 for measuring and treating vergence disorders may comprise a computing device 12 coupled to an augmented reality headset 14 by coupling 18. This coupling 18 may be a network connection, such as a wireless connection through an Internet connection, a 5G connection, a Wi-Fi connection, a Bluetooth connection or the like, wherein the computing device 12 may communicate with and receive communication from the headset 14. The headset 14 is configured to determine and track, independently for each eye, the eye direction (where the eye is looking) and eye movement, using eye tracking technology 16 incorporated into the headset 14, such as the infrared eye tracking technology currently available in such headsets as the HoloLens 2 headset from Microsoft, or the Magic Leap 1 headset from Magic Leap, for example. The computing device 12 may be programmed, such as through an application operating on the device, to perform certain functions in accordance with embodiments of the system 10 in response to user input to the computing device 12 and/or receipt of communication signals and data from the headset 14.

In operation, the headset 14 is first fitted on the patient's head and over the patient's eyes, whereupon calibration of the eye tracking technology 16 may be performed according to calibration procedures native to the headset 14. Calibration is meant to insure that the device accurately measures eye position. It is performed by projecting to the patient, on the headset 14, various geometric targets at various known positions in space. The spatial location of each target is known by the system 12, and the eye position of the patient (of each eye) is determined by the system. This information is used to determine when the subject/patient is fixating on the projected targets. By knowing the position of the target and the measured eye-movements, the system calibrates itself, so that the position of the targets and the alignment of the eyes are mathematically congruent. It is understood that it may be necessary to recalibrate the eye tracking technology 16, as may be appropriate, at other times during use of the system 10.

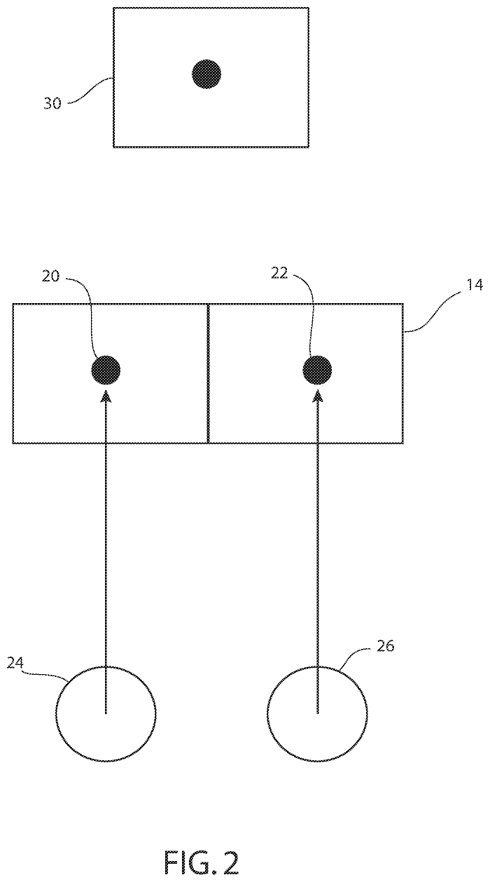

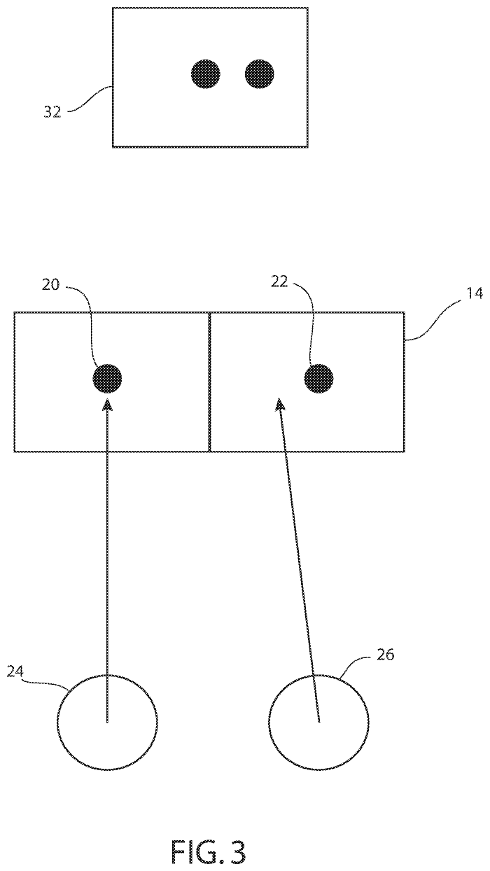

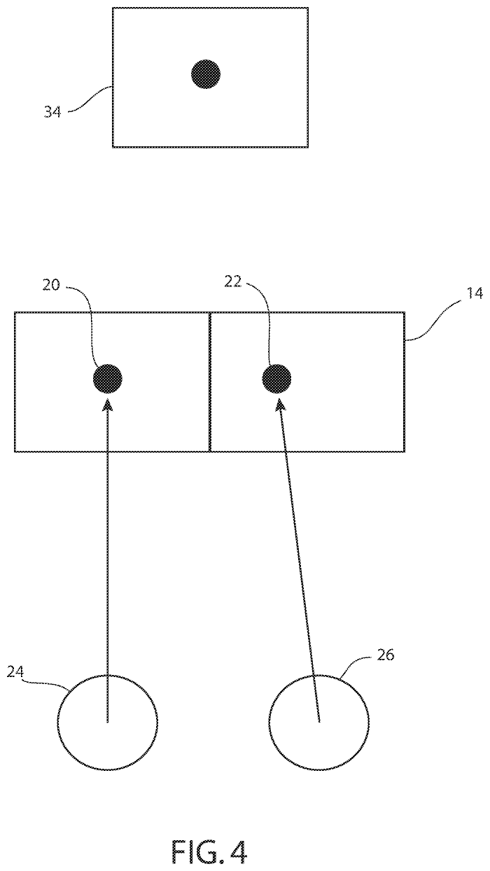

In an embodiment of the system 10, the computing device 12 may be programmed to send for display, on the headset 14, as represented in FIG. 2, a first target image 20 presented to the left eye 24 of the patient and a second target image 22, identical to the first target image 20, is presented to the right eye 26 of the patient. It should be appreciated that in the case of 3D images, the images may be presented with offsets to create retinal disparity, i.e the appearance of stereoscopic vision a.k.a. 3D). The eye tracking technology 16 of the headset 14 determines whether the patient's eyes 24 and 26 are directed to their corresponding target images 20 and 22 using the determined position of each eye and the known position of each target that it is projecting, and communicates eye direction information to the computing device 12 via coupling 18. The arrows in FIGS. 2-4 represent the orientation of the patient's eyes. If the patient's eyes are both aligned on the target, as shown in FIG. 2, then binocular single vision (SBV) will occur, and the patient will see the target images 20 and 22 as a single image, as represented by the image in box 30 of FIG. 2. In some embodiments, if the left eye 24 is on target, and the right eye 26 is not, as shown in FIG. 3, the patient should see double images rather than a single image (the eyes are misaligned) as represented by the image in box 32 of FIG. 3. If this occurs, then according to programming of the computing device 12, the computing device 12 will automatically cause the target image 22 corresponding to the right eye 26 to be moved, either horizontally, vertically, or a combination of horizontally and vertically, until it is realigned with the direction of the right eye 26 (as determined by the known position of the target and the measured position of the eye), as shown in FIG. 4. When this occurs, SBV will occur, as represented by the image in box 34 of FIG. 4. Alternatively, if the right eye 26 is on target, and the left eye 24 is not, then the computing device 12 will automatically cause the target image corresponding to the left eye 24 to be moved, either horizontally, vertically, or a combination of horizontally and vertically, until it is aligned with the direction of the left eye 24 (as determined by the known position of the target and the measured position of the eye). When this occurs, SBV will occur, as represented by the image in box 34 of FIG. 4.

Once SBV has occurred, a vergence demand may then be created by the system 10 by incrementally moving the target images 20 and 22 apart or together, either horizontally, vertically, or a combination of horizontally and vertically. At first, the patient's eyes should normally continue to track their respective targets 20 and 22, at least through small degrees of vergence demand, thereby maintaining SBV. Eventually, however, the vergence demand will become too great for the patient to maintain SBV, at which point one of the patient's eyes will deviate from its target, resulting in double vision (see FIG. 3). The system determines the exact point in time (and target and eye location and direction of gaze for each eye) where this occurs, and logs this information. Note that fusional vergence is the maximum vergence movement enabling SBV (essentially the farthest amount the images 20 and 22 can be separated or moved relative to each from a vergence condition before fusion is lost) and the limit is denoted by the point of diplopia (double vision) (the point at which double-vision occurs). Note that in some situations (such as, for example, where the images must be initially separated in order for the patient to initially achieve BSV due to a phoria or strabismus), the images (20 and 22), which are already separated, may be moved closer to each other to measure the amplitude of fusional vergence or provide treatment. The amplitudes of fusional vergence, in various directions, may thus be measured and stored by the computing device 12.

As noted, it is an advantage of the present invention that the occurrence of either SBV or diplopia, in the fusional vergence amplitude measurement procedure described above, may be determined accurately and automatically (and objectively) by the system 10 using the eye tracking technology 16 of the headset 14, rather than depending on subjective feedback from the patient.

Depending on the data obtained from the above vergence measurements (for example, certain amplitudes of fusional version in a patient), a vision care provider may determine that therapy is indicated in order to improve the amplitudes of fusional version to a more desirable number. For example, motor fusion functions at levels less than clinical norms are often associated with eye-strain or asthenopia. Excessive stress on the vergence system or inability to converge or diverge adequately (in the horizontal direction) or attain supra or infra vergence (in the vertical direction) may warrant therapeutic vergence treatment of the patient.

When therapeutic vergence treatment is warranted, embodiments of the system 10 may be utilized to perform therapeutic vergence treatment exercises to increase amplitudes of fusional vergence. For example, with both eyes of the patient on target (seeing the two independent targets 20 and 22 as one target 30), wherein SBV is initially attained, the system 10 may create a small vergence demand, such as by moving the images 20 and 22 apart, while the position and direction of gaze of the eyes is continuously monitored by the eye tracking technology 16 in conjunction with computing device 12. The degree of vergence demand may be incrementally or continuously increased until the point of diplopia (loss of fusion). In other words, this point of diplopia is when the system objectively determines, using the measured eye position, direction of gaze and known target positions 20 and 22, that the patient is now seeing two images rather than one. As noted above, the system 10 tracks both the time at which this occurs, and the position and direction of gaze of each eye 24 and 26 and each target 20 and 22, and stores this information. Once diplopia occurs (loss of fusion), as determined by using the eye tracking technology 16 (to determine each eye's position and direction of gaze) and known target positions 20 and 22, the computing device 12 may be programmed to automatically bring the images 20 and 22 together again until the point at which SBV is again attained. Again, the point at which SBV is regained is determined objectively by the system, using the eye position data from the headset 14 and known target positions 20 and 22 for each eye. This data, along with the time of regaining SBV, are stored by the system. Then, in order to continue exercising the eyes 24 and 26, the images 20 and 22 may again be incrementally or continuously increased again until a new point of diplopia is reached. By repetition of this exercise for a certain duration or number of iterations, the amplitude of fusion is gradually increased until SBV can be comfortably achieved by the patient to within a predetermined range of acceptable values. It is understood that similar vergence exercises may be performed by the system 10 automatically moving the target images 20 and 22 closer together instead of apart, and doing this in an iterative process, and that vergence may be in the horizontal direction, vertical direction, or a combination of horizontal and vertical directions. This repetitive process is designed to improve the vergence reflexes in patients.

Alternatively, a vision care provider, on reviewing the stored fusional vergence data and amplitude of fusional vergence, may direct the system regarding the duration or number of iterations of exercise, and/or the magnitudes of increasing/decreasing the separation of images, the duration of time between iterations, and various other parameters of the therapy. In practice, therapy sessions may typically be engaged at spaced intervals, over days, weeks, or months until the desired level of improvement is attained, based on the objective patient data determined and stored by the system.

As noted above regarding the fusional vergence amplitude measurement procedure, it is an advantage of the present invention that the occurrence of either SBV or diplopia, in the therapeutic vergence treatment procedure, as described herein, may be determined accurately, automatically and objectively by the system 10 using the eye tracking technology 16 of the headset 14, rather than depending on subjective feedback from the patient to tell the system when fusional vergence occurs or is lost.

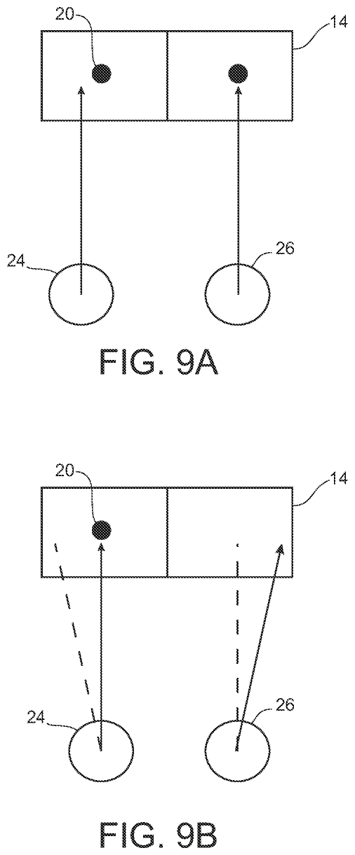

As noted above, the system may also be used to objectively determine strabismus and phoria characteristics of a patient's eyes. In this case, an identical object (target) is presented to each eye 24 and 26 (see FIG. 9a). The system determines the exact position and direction of gaze of each of the eyes 24 and 26 using the eye tracking technology 16 of the headset 14, which communicates the eye position and direction of gaze to the system. Next, (see FIG. 9b) the right image 22 is eliminated (blanked) for a short period of time (for example, 2 seconds), so that only the left eye is presented the target. If, when the right eye is blanked, the left eye moves to fixate on the target being presented to it (as determined by the system continuously monitoring the position of the left eye), the amount of movement (turn of the eye) is determined by the system (based on the system's knowledge of the initial position of the eye and the final, fixated position of the eye). This amount of movement is captured and logged by the system, as is the fact that a strabismus condition exists for the left eye. If, however, the system determines that the left eye did not need to move in order to be fixated on the target, the system determines if the right (blanked) eye moved from its initial position when it was blanked. If it did move, the magnitude of that movement is determined by the system (based on the initial and final position of the right eye as determined by the system), and the system captures that a phoria exists for the right eye (and the magnitude of that phoria based on the difference between the initial and final positions of the right (blanked) eye.

Next, the above process is repeated, but blanking the left eye rather than the right eye. As above, an identical object (target) is presented to each eye. The system determines the exact position of each of the eyes. Next, the left image is eliminated (blanked), so that only the right eye is presented the target. If, when the left eye is blanked, the right eye moves to fixate on the target being presented to it (as determined by the system continuously monitoring the position of the right eye), the amount of movement (turn of the eye) is determined by the system (based on the system's knowledge of the initial position of the eye and the final, fixated position of the eye). This amount of movement is captured and logged by the system, as is the fact that a strabismus condition exists for the right eye. If, however, the system determines that the right eye did not need to move in order to be fixated on the target, the system determines if the left (blanked) eye moved. If it did move, the magnitude of that movement is determined by the system (based on the initial and final position of the left eye as determined by the system), and the system captures that a phoria exists for the left eye (and the magnitude of that phoria based on the difference between the initial and final positions of the left (blanked) eye.

It should be appreciated that both vertical and horizontal measurements may be taken in both cases in determining the existence (and magnitude) of phoria or strabismus conditions.

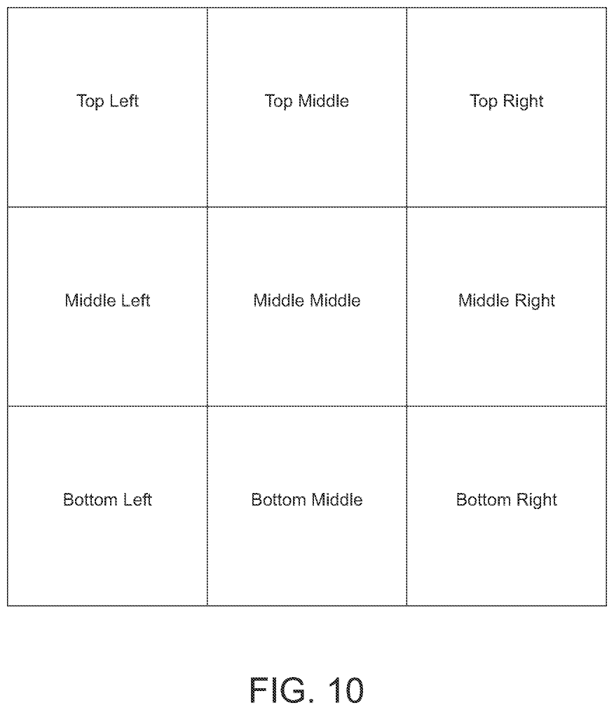

In an alternative embodiment, called, for example, “motor field”, the above procedures (measurement and treatment phoria and/or amount of strabismus) may be performed through a number of gaze positions. For example, in one embodiment, 9 positions are utilized: top left, top middle, top right, middle left, middle middle, middle right, bottom left, bottom middle, bottom right (see, e.g. FIG. 10). In this embodiment, the projected images for each of the procedures are projected successively at each of the 9 gaze positions, and data gathered (and or treatment administered) at that particular gaze position. For example (with respect to phoria or strabismus evaluation and treatment), the images are first projected at the first gaze position (for example, top left), such that the images appears to be one image. Next, the image is “blanked” for one of the eyes (for example, the right eye) for a short period of time. When this occurs, the above-noted phoria and strabismus measurements are taken by the system. That data is stored by the system. Next, the same is done for the left eye (the left eye is blanked for a brief period, the eye positions are measured, and the data is also stored. Next, the system moves the images to the next field position (say, for example, top middle), and the same process is repeated, saving all the measurements for that position. The same process is repeated for each of the 9 gaze positions, and the data is stored for each of those positions. The data for each position may be automatically graphed based on the stored data. This data may then be utilized by the vision care provider to determine if the patient has any vision issues (for example, paresis or other non-comitancy), and to determine an appropriate treatment plan for those vision issues.

As can be seen, the system provides for objective measurement vergence, strabismus and phoria conditions using independent eye movement measurements. In an alternative embodiment, with regard to phoria and strabismus measurements, rather than extinguishing the right or left target, a white amorphous visual field may be substituted (in other words, the fusible target for the eye to be blanked is replaced by a white field with no fusible detail.

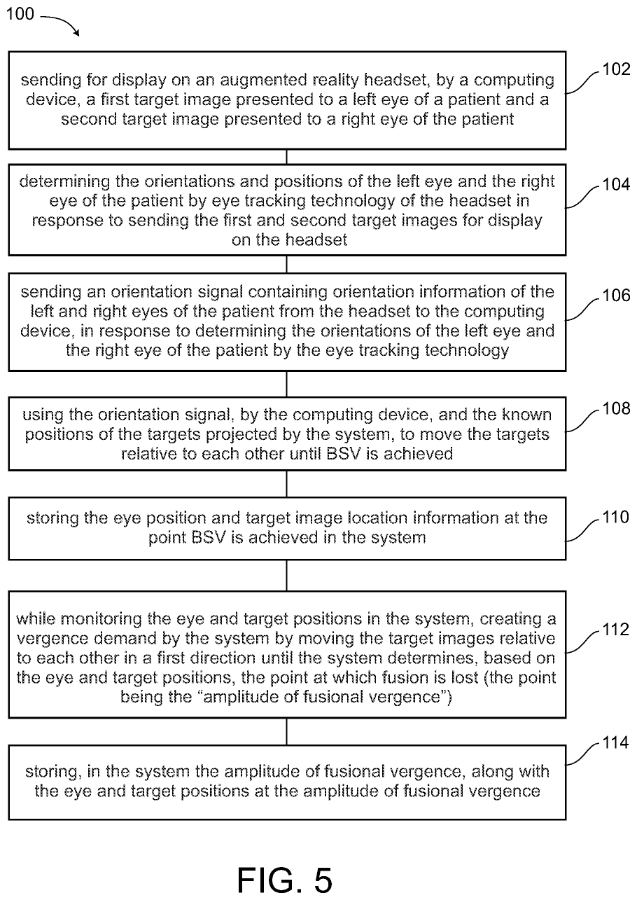

Referring to the drawings, FIG. 5 illustrates a block diagram of steps of a method 100 of using an augmented reality-based system for measuring and treating vergence disorders, according to an embodiment, the method 100 comprising the steps of: sending for display on an augmented reality headset, by a computing device, a first target image presented to a left eye of a patient and a second target image presented to a right eye of the patient [Step 102]; determining the orientations and positions of the left eye and the right eye of the patient by eye tracking technology of the headset in response to sending the first and second target images for display on the headset [Step 104]; sending an orientation signal containing orientation information of the left and right eyes of the patient from the headset to the computing device, in response to determining the orientations of the left eye and the right eye of the patient by the eye tracking technology [Step 106]; using the orientation signal, by the computing device, and the known positions of the targets projected by the system, to move the targets relative to each other until BSV is achieved [Step 108]; storing the eye position and target image location information at the point BSV is achieved in the system [Step 110]; while monitoring the eye and target positions in the system, creating a vergence demand by the system by moving the target images relative to each other in a first direction until the system determines, based on the eye and target positions, the point at which fusion is lost (the point being the “amplitude of fusional vergence” [Step 112]; storing, in the system the amplitude of fusional vergence, along with the eye and target positions at the amplitude of fusional vergence [Step 114];

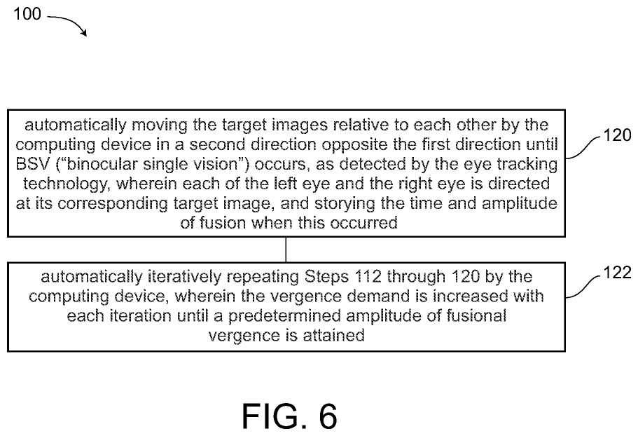

In some embodiments, as further illustrated in the block diagram of FIG. 6, the method 100 may further comprise: automatically moving the target images relative to each other by the computing device in a second direction opposite the first direction until BSV (“binocular single vision”) occurs, as detected by the eye tracking technology, wherein each of the left eye and the right eye is directed at its corresponding target image, and storying the time and amplitude of fusion when this occurred [Step 120]; and automatically iteratively repeating Steps 112 through 120 by the computing device, wherein the vergence demand is increased with each iteration until a predetermined amplitude of fusional vergence is attained [Step 122].

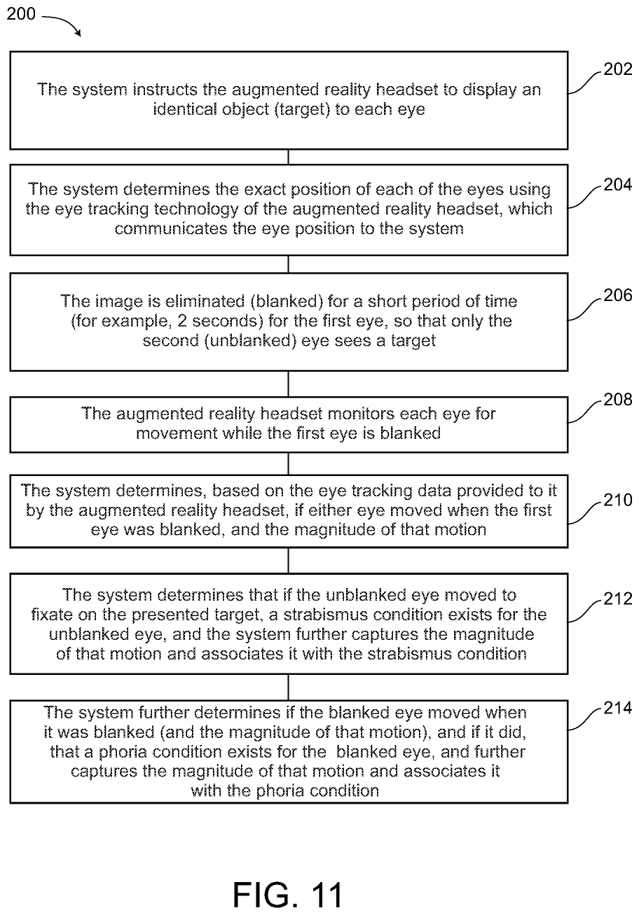

In another embodiment, a method 200, generally illustrated in FIG. 11 is provided to objectively determine phoria and strabismus conditions of a patients' eyes. The method comprises the following steps: The system instructs the augmented reality headset to display an identical object (target) to each eye such that BSV occurs [202]; The system determines the exact position of each of the eyes using the eye tracking technology of the augmented reality headset, which communicates the eye position to the system [204]; The image is eliminated (blanked) for a short period of time (for example, 2 seconds) for the first eye, so that only the second (unblanked) eye sees a target [206]; The augmented reality headset monitors each eye for movement while the first eye is blanked [208]; The system determines, based on the eye tracking data provided to it by the augmented reality headset, if either eye moved when the first eye was blanked, and the magnitude of that motion [210]; The system determines that if the unblanked eye moved to fixate on the presented target, a strabismus condition exists for the unblanked eye, and the system further captures the magnitude of that motion and associates it with the strabismus condition [212]; The system further determines if the blanked eye moved when it was blanked (and the magnitude of that motion), and if it did, that a phoria condition exists for the blanked eye, and further captures the magnitude of that motion and associates it with the phoria condition [214]; The system then repeats the process beginning with step 202, but switching the eye that is blanked.

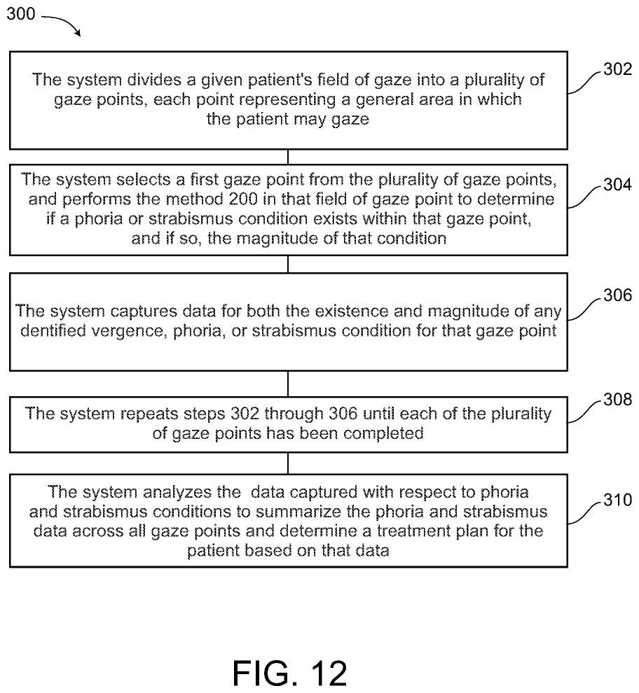

In yet another embodiment, a method 300, generally illustrated in FIG. 12 is provided to objectively determine phoria and strabismus conditions of a patients' eyes through multiple gaze potions in a field of gazes. The method comprises the following steps: The system divides a given patient's field of gaze into a plurality of gaze points, each point representing a general area in which the patient may gaze [302]; The system selects a first gaze point from the plurality of gaze points, and performs the method 200 in that field of gaze point to determine if a phoria or strabismus condition exists within that gaze point, and if so, the magnitude of that condition [304]; The system captures data for both the existence and magnitude of any identified vergence, phoria, or strabismus condition for that gaze point [306]; The system repeats steps 302 through 306 until each of the plurality of gaze points has been completed [308]; The system analyzes the data captured with respect to phoria and strabismus conditions to summarize the phoria and strabismus data across all gaze points and determine a treatment plan for the patient based on that data [310]

In an alternative embodiment, both the position of each eye's target and the objectively measured position/orientation of each of the patient's eyes is simultaneously displayed on a separate display device visible to the vision care provider, along with indicators of when fusion has occurred or been lost, allowing the vision care provider to view in real-time what the patient is seeing and how the patient's eyes are responding. This can aide the vision care provider in adjusting parameters of the system (how much stress to subject the eyes to when incrementally separating targets to induce loss of fusion, duration between iterations, etc.). In addition, this allows the vision care provider to see if the patient is looking at the target.

In an alternative embodiment, when the system determines that there has been loss of fusion, it waits a predetermined amount of time (without moving the targets) to determine if the patient is able to regain fusion on their own. Once that time period has passed, and fusion has not been regained, the system will gradually move the targets back toward each other until fusion is regained, capturing both eye position and target position data through the process.

In an alternative embodiment, the system user can adjust the speed at which targets are moved relative to each other, and/or the amount of time that a patient is permitted to use the system to exercise the eyes through the iterative process discussed above.

In an alternative embodiment, the system stores, for each patient, the precise point at which fusion is lost when targets are separated, and, in therapy sessions, causes the targets to immediately be separated to that point of loss of fusion (rather than incrementally separating targets from a point of fusion), and continuously increments vergence demands away from that point, and back to that point, iteratively, to work the eye muscles right at the edge of loss of fusion.

In an alternative embodiment, input from an input device is utilized by the patient to demand separation of the targets, or to move targets together.

It should be appreciated that in the present invention, the system is able to determine, automatically and objectively, and without subjective patient input, which of the patient's eyes is not aligned to the target, unlike previous systems where this cannot be determined.

It should also be appreciated, that in the present system, the distance between the eyes and the targets is fixed, providing a reliable measurement of the parameters discussed above, whereas in previous systems, displaying images on a monitor where the distance between the patient's eyes and targets varies based on any head or body movement of the patient, decreasing the reliability of any system measurements.

As noted, this system provides objected data, rather than data determined by subjective input that may vary based on the age of the patient, attention span, length of time between when the patient realizes fusion has been lost and when it was actually lost, duration of time between patient perception of fusion loss and communication of that via patient manual input, honesty of patient, and other subjective factors.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire-line, optical fiber cable, RF, etc., or any suitable combination of the foregoing.

Computer program code for carrying out operations for aspects of the present invention may be written in any combination of one or more programming languages, including an object-oriented programming language such as Java, Unity, Smalltalk, C++, or the like, and conventional procedural programming languages, such as the “C” programming language or similar programming languages. The program code may execute entirely on the computing device 12, as a stand-alone software package, partly on the computing device 12 and partly on a remote computer, or entirely on the remote computer or server. In the latter scenario, the remote computer may be connected to the computing device 12 through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, cloud-based infrastructure architecture, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

1. A system for measuring and treating eye disorders, comprising:
a wearable headset configured to be positioned on an eye patient's head and independently display objects to each of a patient's eyes responsive to received signals;
eye tracking technology incorporated into the headset and configured to determine, independently, for each eye, the position and direction of gaze of each of a patient's eyes;
a computing device connected to the headset by means of a coupling, the computing device comprising firmware configured to cause images of objects to be communicated to each eye, via the coupling, at specific, pre-determined positions for each eye, the computing device further configured to receive, from the eye tracking technology of the headset, eye position and direction of gaze data, and further configured, based on the data and position of the images of objects, a clinical condition of the patients eyes.

2. The system of claim 1, wherein the clinical condition is binocular singular vision.

3. The system of claim 1, wherein the clinical condition is the existence of a strabismus condition.

4. The system of claim 3, wherein the clinical condition is the magnitude of a strabismus condition.

5. The system of claim 1, wherein the clinical condition is the existence of a phoria condition.

6. The system of claim 5, wherein the clinical condition is the magnitude of a phoria condition.

7. The system of claim 1, wherein the position of the objects is varied, over time, by the system, to alter a clinical condition.

8. The system of claim 7, wherein the duration or magnitude of the variance of the position of the objects is determined and varied, by the system, based on the magnitude of a clinical condition determined by the system.

9. A method for the measurement and treatment of eye conditions, comprising the steps of:
sending for display on an augmented reality headset, by a computing device, a first target image presented to a left eye of a patient and a second target image presented to a right eye of the patient;
determining the orientations and positions of the left eye and the right eye of the patient by eye tracking technology of the headset in response to sending the first and second target images for display on the headset;
sending an orientation signal containing orientation information of the left and right eyes of the patient from the headset to the computing device, in response to determining the orientations of the left eye and the right eye of the patient by the eye tracking technology;
using the orientation signal, by the computing device, and the known positions of the targets projected by the system, to move the targets relative to each other until BSV is achieved;
storing the eye position and target image location information at the point BSV is achieved in the system;
while monitoring the eye and target positions in the system, creating a vergence demand by the system by moving the target images relative to each other in a first direction until the system determines, based on the eye and target positions, the point at which fusion is lost (the point being the “amplitude of fusional vergence”; and,
storing, in the system the amplitude of fusional vergence, along with the eye and target positions at the amplitude of fusional vergence;

10. The method of claim 9, further comprising the steps of:
automatically moving the target images relative to each other by the computing device in a second direction opposite the first direction until BSV (“binocular single vision”) occurs, as detected by the eye tracking technology, wherein each of the left eye and the right eye is directed at its corresponding target image, and storying the time and amplitude of fusion when this occurred; and,
automatically iteratively repeating the step of creating a vergence demand and subsequent steps, wherein the vergence demand is increased with each iteration until a predetermined amplitude of fusional vergence is attained.

11. A method for measuring and treating eye disorders comprising the steps of:
instructing an augmented reality headset to display an identical object (target) to each eye such that BSV occurs in response to operation of a system for measuring and treating eye disorders;
determining by the system an exact position of each of the eyes using an eye tracking technology of the augmented reality headset, which communicates the eye position to the system;
Eliminating (blanking) the object (target) for a short period of time for the first eye, so that only the second (unblanked) eye still sees the target;
monitoring each eye for movement while the first eye is blanked using the augmented reality headset;
determining by the system, based on eye tracking data provided to the system by the augmented reality headset, if either eye moved when the first eye was blanked, and the magnitude of that motion;
determining by the system that if the unblanked eye moved to fixate on the presented target, a strabismus condition exists for the unblanked eye, and the system further captures the magnitude of that motion and associates it with the strabismus condition;
determining by the system if the blanked eye moved when it was blanked (and the magnitude of that motion), and if it did, that a phoria condition exists for the blanked eye, and further captures the magnitude of that motion and associates it with the phoria condition; and, repeating the steps by the system, but switching the eye that is blanked.

12. The method of claim 11, further comprising the step of repeating the method, for each eye, at a plurality of gaze positions.

13. The method of claim 12, wherein the number of the plurality of gaze positions is at least 3.
